(19) United States
(12) Patent Application Publication
(10) Pub. No.: US 2021/0057088 A1
(43) Pub. Date: Feb. 25, 2021

(54) TOUCHLESS AND AUTOMATIC METHOD TO INITIATE, TRACK AND CONTROL WITH VERIFICATIONS, BIOMETRICS AND GPS PRE-OPERATIVE SURGICAL PROCEDURES AND ACTIVITIES, DAY OF SURGERY PROCEDURES AND ACTIVITIES, AND POST-OPERATIVE SURGICAL PROCEDURES AND ACTIVITIES

(71) Applicant: LAUREN BROWNFIELD, Houston, TX (US)

(72) Inventor: LAUREN BROWNFIELD, Houston, TX (US)

(21) Appl. No.: 17/091,161

(22) Filed: Nov. 6, 2020

Related U.S. Application Data
(63) Continuation-in-part of application No. 16/389,186, filed on Apr. 19, 2019, now abandoned.
(60) Provisional application No. 62/674,153, filed on May 21, 2018.

(51) Int. Cl. G16H 40/20; G16H 20/10; G16H 20/40; G16H 10/60; G06Q 10/02; G16H 40/67; G16H 70/20; G06Q 10/10; G16H 50/20; G16H 50/70; H04L 29/06; A61B 5/00; A61B 5/11

(52) U.S. Cl. CPC G16H 40/20; G16H 20/10; G16H 20/40; G16H 10/60; G06Q 10/02; G16H 40/67; A61B 5/1112; G06Q 10/109; G16H 50/20; G16H 50/70; H04L 63/0861; A61B 5/4833; G16H 70/20

(57) ABSTRACT

A method for treating a patient using patient data and surgical reservation data to identify confirm with a medical provider and transmit surgical reservation data with pre-operative instructions and pre-operative medications; day of surgery instructions with day of surgery medications; post-operative instructions with post-operative medications using an administrative processor connected to a medical provider client device and a patient client device via a network to automatically confirm surgeries, medicines, pharmacies, and provide reminders to institute preoperative, day of surgery and post-operative surgery instructions.

### CROSS REFERENCE TO RELATED APPLICATION

The present application claims priority to and the benefit of co-pending U.S. Provisional Patent Application Ser. No. 62/674,153 filed on May 21, 2018, entitled “SYSTEM FOR USE WITH ONE OR MORE SOURCES OF PATIENT DATA” and U.S. Utility patent application Ser. No. 16/389,186 filed on Apr. 19, 2019, entitled “SYSTEM FOR USE WITH ONE OR MORE SOURCES OF PATIENT DATA.” These references are hereby incorporated in their entirety.

### FIELD

The present embodiment generally relates to a method for use with one or more sources of patient-affiliated data, corresponding with a group of patients who have each scheduled a surgery and may have been prescribed a pre-surgery instruction and medication, a day of surgery instruction and medication, and a post-operative instruction and medication.

### BACKGROUND

A need exists for a touch free automatic method for use with one or more sources of patient-affiliated data, corresponding with a group of patients who have each been scheduled a surgery with easy to understand reminders to prevent death during or after a surgery.

The present embodiments meet these needs.

### BRIEF DESCRIPTION OF THE DRAWINGS

The detailed description will be better understood in conjunction with the accompanying drawings as follows:

FIG. 1 depicts a diagram of equipment used in the method.

FIGS. 2A-2E depicts rules in the administrative computer readable memory usable with the method.

FIGS. 3A-3C shows a sequence of steps according to the method.

FIG. 4 shows a Patient Client Device memory.

FIG. 5. Depicts a Medical Provider Client Device.

The present embodiments are detailed below with reference to the listed Figures.

### DETAILED DESCRIPTION OF THE EMBODIMENTS

Before explaining the present method in detail, it is to be understood that the method is not limited to the particular embodiments and that it can be practiced or carried out in various ways.

The invention relates to a touchless and automatic method to initiate, track and control with verifications, biometrics and GPS, pre-operative surgical procedures and activities, day of surgery procedures and activities, and post-operative surgical procedures and activities.

The method for treating a surgical patient is used simultaneously by medical providers with medical provider client devices and patients with patient client devices that connect via a network, such as a cellular network, using secure biometric file matching and GPS location matching with an administrative processor to match patient data to surgical reservation data and automatically transmit surgery appropriate confirmation of surgical reservation data, pre-operative surgical procedures and activities, day of surgery procedures and activities, and post-operative surgical procedures and activities via the network bidirectionally to and from a patient client device and a medical provider client device and an administrative processor. Instructions stored in the memory of the administrator match stored biometric files and surgical reservation data with patient data stored in the memory of the administrative processor to build and transmit a sequence of messages each requiring verification from the medical provider client device and the patent client device. The message consist of: (i) pre-operative instructions and pre-operative medications, (ii) information on the category of surgery selected by a medical provider using a library of categories of surgeries in the computer readable administrative processor memory; (iii) day of surgery instructions with day of surgery medications; and (iv) post-operative instructions with post-operative medications which instructions and medications are stored in the computer readable administrative memory.

The automatically custom built messages are then transmitted from the administrative processor at calculated timed intervals based on a formulation stored in the computer readable administrative processor memory that includes the date of surgery and pre-operative lead time to the surgery and post-operative recovery time.

The administrative processor is in communication with the patient client device and medical provider client device via a network and the medical provider client device and the patient client device uses custom and over the counter application installed on those devices to automatically confirm surgeries using biometric files, confirm pre-operative and day of surgery medicines using biometric verifications, confirm pharmacies in the patient data using verifications, and provide reminders of any pre-operative, day of surgery, and post-operative surgery instructions to the patient client device with verifications that they read the instructions.

The invention stops death by preventing patients from continuing concurrent medications that could cause complications in surgery.

The inventions stops death by preventing people from bleeding out due to complications from surgery by stopping pre surgery the use of blood thinners.

The invention stops suicide by preventing people from taking too many opioids or certain opioids due to allergies or other concurrent medication consumption.

The invention prevents death by repeatedly and automatically generating messages in the form of requests for post-operative appointments so that a qualified medical professional can assess wound healing, preventing sepsis and prevent amputations.

The following terms shall be used:

The term “administrative computer readable memory “refers to data storage which is non-evanescent and configured to store sets of rules and logic for use by a processor.

The term “confirmation of surgical reservation data” refers to a transmittal to the patient of the date and time and details of the surgery and the return reply by the patient agreeing to the surgery reservation data.

The term “database for containing the patient data” refers to a relational interactive database.

The term “library of categories of surgeries” refers to a database of surgery information which includes but is not limited to pre-operative instructions such as “start antibiotics 3 days before surgery” or “stop aspirin 5 days before surgery”. The Database can include lists of pre-operative medications such as Ibuprofen, Amoxicillin with dosage amounts and times for taking dosages given dosage amounts. The Library of categories of surgeries include; day of surgery instructions such as “do not eat within 6 hours of surgery” and day of surgery medications such as “take Augmentin twice a day after surgery; and post-operative instructions such as “don't drive the car until the following day after surgery” and “eat soft food” or “don't blow nose” with post-operative medications such as take TYLENOL3™ daily until pain dissipates.

The term “library of pharmacies” refers to a database of local and national pharmacies with addresses, names, phone numbers and fax numbers for communication of patient information.

The term “medical provider” refers to a name of a doctor, names of medical personnel working for the doctor.

The term “network” refers to a local area network, a wide area network, a cellular network, a satellite network, or a global communication network.

The term “patient client device” can be a smart phone, a tablet, and iPad, a wearable computer, including a smart watch, a laptop, or a desk top.

The term “patient data” includes but is not limited to applicable pre-surgery medical condition of a patient such as the patient has type II Diabetes; a preferred pharmacy such as WALGREENS™; all concurrent medicine such as aspirin, SYNTHROID™ high blood pressure medicine, and the like not related to the surgery, and includes surgical reservation data.

The term “patient identification number” refers to an automatically generated number from the patient surgery application when a patient desires to join the structured set of data held in computer memory that is accessible in various ways.

The term “patient surgery application” refers to a set of rules to manage pre-surgery, day of surgery and post-operative surgery patient procedures, medications, and patient instructions.

The term “personal identification number” shall include alpha numeric combinations, all numeric combinations, all alpha combinations, biometric files including retinal images and voice prints.

The term “post-operative instructions” refers to a set of rules that a patient must comply with based on a specific surgery at a pre-determined time including but not limited to medications required to be taken or avoided, patient behaviors modifications, such as not drinking, and can include scheduling and attending post-operative appointments and automatically transmitting reminders to attend a post-operative appointment or call the office to reschedule the appointment.

The term “preset time intervals” refers to specific time and dates relative to a patient surgery reservation data and surgery date and time using a highly precise clock.

The term “preferred pharmacy” refers to a patient selected pharmacy that may or may not be in the library of pharmacies.

The term “prewritten message” refers to a set of words transmitted to a client device to asking the patient to perform a specific behavior.

The term “processor” refers to a server, a cloud based server, or any device in communication with computer readable memory can compute according to a set of defined rules, and may include a laptop, a tablet, or series of laptops in communication with each other.

The term “surgical reservation data” includes a date, a time, a location, a parking garage location and pricing and the name of the doctor; and a patient digital address includes an email, a phone number or an iMESSAGE™ address.

The term “verification window” refers to a display screen with a prewritten set of words asking the patient or medical provider to verify that the information is correct regarding medications or other instructions.

More specifically, the method is a touchless and automatic method to set up a plurality of patients for surgery simultaneously by touchlessly combining patent data and custom messaging and verification of messaging using biometric data for preoperative procedures including pre-operative medication, GPS data for touchless day of surgery check in, messaging and verification of messaging using biometric files concerning day of surgery medication, and messaging with biometric verification of post-operative procedures including post-operative medication.

The steps of the method include the following including connecting an administrative processor to a network.

The next step involves connecting a patient client device to the network and the administrative processor.

The patient client device has a client device memory configured to:receive and store patient data including patient name, patient identification number, a preferred pharmacy of a patient, pre-surgery medical condition of a patient; patient concurrent medicine; patient surgical reservation data; and a patient digital address of a patient; receive and store instructions to receive and transmit information bi-directionally patient data including biometric files of the patient and GPS data from the patient client device to the administrative processor.

The next step involves connecting a medical provider client device to the network and the administrative processor.

The medical provider client device has a client device memory configured to: receive store and update a Library of Categories of Surgery; receive and store patient surgical reservation data, and receive and store instructions to receive patient data and customize automatically built messages by the administrative processor for transmission to the patient client device.

Next, an administrative computer readable memory is connected to the administrative processor.

The administrative computer readable memory then receives patient data from the patient client device to the administrative computer readable memory.

The administrative computer readable memory receives and stores: patient data consisting of patient name, patient identification number, a preferred pharmacy of a patient, pre-surgery medical condition of a patient; patient concurrent medicine; patient surgical reservation data; and a patient digital address of a patient.

The administrative computer readable memory stores a Library of Categories of Surgeries, with each Category of Surgery comprising: pre-operative instructions with pre-operative medications; day of surgery instructions with day of surgery medications; post-operative instructions with post-operative medications; and a library of pharmacies.

The administrative computer readable memory stores and then implements many sets of rules which instruct the administrative processor to act sequentially including as identification rules that cause the administrative processor to automatically generate a patient identification number and link the patient identification number to the patent data, and invitation rules that cause the administrative processor to automatically use the patient identification number to transmit an invite to a surgery to a patient surgery application that has been downloaded into the patient client device, wherein the invitation is in the form of one of a plurality of prewritten messages requesting a personal identification number.

The administrative computer readable memory causes the administrative processor to use matching rules that cause the administrative processor to automatically link the personal identification number to the patient data.

The administrative computer readable memory causes the administrative processor to use preferred pharmacy rules that cause the administrative processor to automatically confirm a preferred pharmacy of the patient's choice using the patient data and a Library of Pharmacies stored in the administrative computer readable memory.

The administrative computer readable memory causes the administrative processor to use preoperative instructions rules that cause the administrative processor to automatically transmit pre-operative instructions with pre-operative medications from the Library of Categories of Surgeries to the medical provider client device using a Category of Surgery selected by the medical provider from the Library of Categories of Surgeries for review and customization by the medical provider.

The administrative computer readable memory causes the administrative processor to use first verification rules that cause the administrative processor to automatically present a first verification window to the medical provider client device using biometrics to verify acceptance of the selected pre-operative instructions with pre-operative medications and prompting the medical provider to customize the pre-operative instructions with pre-operative medications prior to actuating any subsequent steps

The administrative computer readable memory causes the administrative processor to use day of surgery rules that cause the administrative processor to automatically transmit day of surgery instructions with day of surgery medications to the medical provider client device.

The administrative computer readable memory causes the administrative processor to use second verification rules that cause the administrative processor to automatically present a second verification window to the medical provider client device requesting acceptance of the day of surgery instructions with day of surgery medications using a biometric file and requesting the medical provider to customize the day of surgery instructions with day of surgery medications prior to actuating any subsequent steps.

The administrative computer readable memory causes the administrative processor to use post-operative rules that cause the administrative processor to automatically transmit post-operative instructions with post-operative medications to a medical provider client device.

The administrative computer readable memory causes the administrative processor to use third verification rules that cause the administrative processor to automatically present a third verification window to the medical provider client device requesting acceptance of the post-operative instructions with post-operative medications using a biometric file and requesting the medical provider to customize the post-operative instructions with post-operative medications prior to actuating any subsequent steps.

The administrative computer readable memory causes the administrative processor to use preset message transmission rules that cause the administrative processor to automatically transmit a preset message stored in the Library of Categories of Surgery to the patient client device that surgical reservation data can be viewed.

The administrative computer readable memory causes the administrative processor to use personal identification rules that cause the administrative processor to receive and store a personal identification number in the administrative computer readable memory enabling the patient client device to access surgical reservation data.

The administrative computer readable memory causes the administrative processor to use fourth verification rules that cause the administrative processor to automatically present a fourth verification window to the patient client device enabling a patient to verify acceptance of the surgical reservation data and preferred pharmacy using a biometric file.

These steps are used to (i) touchlessly register a patient for surgery, (ii) touchlessly receive patient data, (ii) remotely build custom messaging to a patient with verification of messaging using biometric data and patient GPS data from a patient client device for (a) remotely transmitting and verifying preoperative procedures including pre-operative medications, (b) touchlessly registering a patient for a day of surgery using GPS coordinates from the patient client device, (c) touchlessly registering a patient for day of surgery procedures using GPS coordinates from the patient client device (d) automatically transmitting and verifying post-operative procedures (e) automatically messaging pharmacies and patients and medical providers regarding preparation of, time for pick up of and verification of pick-up of pre-surgery medication, day of surgery medication and post-surgery medication, and (f) automatically and remotely messaging the patient and medical provider verification from the patient client device that a patient is taking the designated medication.

The method includes using a set of pre-operative customization rules in the administrative processor computer readable memory 40 to cause the administrative processor to automatically generate access to patient pre-operative instructions with patient specific pre-operative medications 63 and customize pre-operative instructions in view of all concurrent medicine 34 of the patient when surgical reservation data is accessed

The method includes using a set of day of surgery rules in the computer readable memory to cause the administrative processor to automatically suggest the medical provider customize patient specific day of surgery instructions with day of surgery medications; and enable customization of patient specific post-operative instructions with post-operative medications when surgical reservation data is accessed.

The method includes using a set of “medication is ready” rules in the computer readable memory to instruct the administrative processor to automatically transmit a message to a patient client device when at least one of: a pre-operative medication, day of surgery medication, and a post-operative medication is ready for pick up at the preferred pharmacy.

The method can include using a set of non-surgery preoperative reminder rules in the computer readable memory to instruct the administrative processor to automatically transmit a message to a patient client device based on preset time intervals notifying a patient of day of surgery non-medication activity.

The method can include a set of changes in concurrent medication rules in the computer readable memory to instruct the administrative processor to automatically transmit a message to a patient client device notifying a patient of changes in concurrent medications.

The method can include a set of “need to contact medical provider” rules in the computer readable memory to cause the administrative processor to automatically transmit a message to a patient client device notifying a patient to immediately contact the medical provider with questions after surgery.

The method can include a set of “responsible party” rules in the computer readable memory to cause the administrative processor to automatically transmit a message to a patient client device reminding a patient to share post-operative instructions with a responsible party.

The method can include a set of “start pre-operative medication” rules in the computer readable memory to cause the administrative processor to automatically transmit a message to a patient client device reminding a patient to start pre-operative medications based on preset time intervals.

The method can include a set of “transmit a message on day of surgery” rules in the computer readable memory to cause the administrative processor to automatically transmit a message to a patient client device reminding a patient to start day of surgery medications based on preset time intervals.

The method can include a set of post-operative medication rules in the computer readable memory to cause the administrative processor to automatically transmit a message to a patient client device reminding a patient to start post-operative medications based on preset time intervals.

The method can include a set of dosage verification rules in the computer readable memory to cause the administrative processor to automatically transmit a message to a patient client device reminding a patient to take a dosage of a certain medication by time of day and to present a verification window to the patient client device enabling the patient to verify that the patient took the dosage of the medication and the time of day that the patient took the dosage.

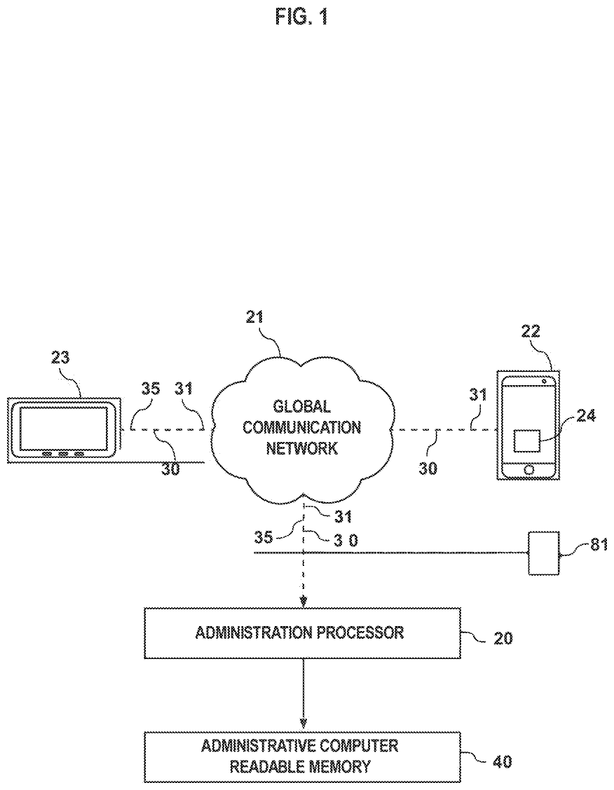

Turning now to the Figures, FIG. 1 shows a step of the method wherein one or more sources of patient-affiliated data are utilized. The patient affiliated data corresponds to a group of patients having a surgery and are individually prescribed at least one of: (i) a pre-operative medication, (ii) a day of surgery medication, and (iii) a post-operative medication.

The method uses an administrative processor 20 connected to a network 21 and connected to a patient client device 22 with patient client device memory 24 to cause the receipt and transmission of information bi-directionally to a patient client device, which can include patient data 30 and to cause the transmission of pre-operative instructions, day of surgery instructions, and post operative instructions to the patient client device.

The method requires use of an administrative computer readable memory 40 connected to the administrative processor.

The method uses a medical provider client device 23 connected to the network and to the administrative processor to transmit information bi-directionally to and from the administrative processor as well as to receive patient data 30, and to input surgical reservation data 35 to the administrative processor and to receive on confirmation of surgical reservation data 31.

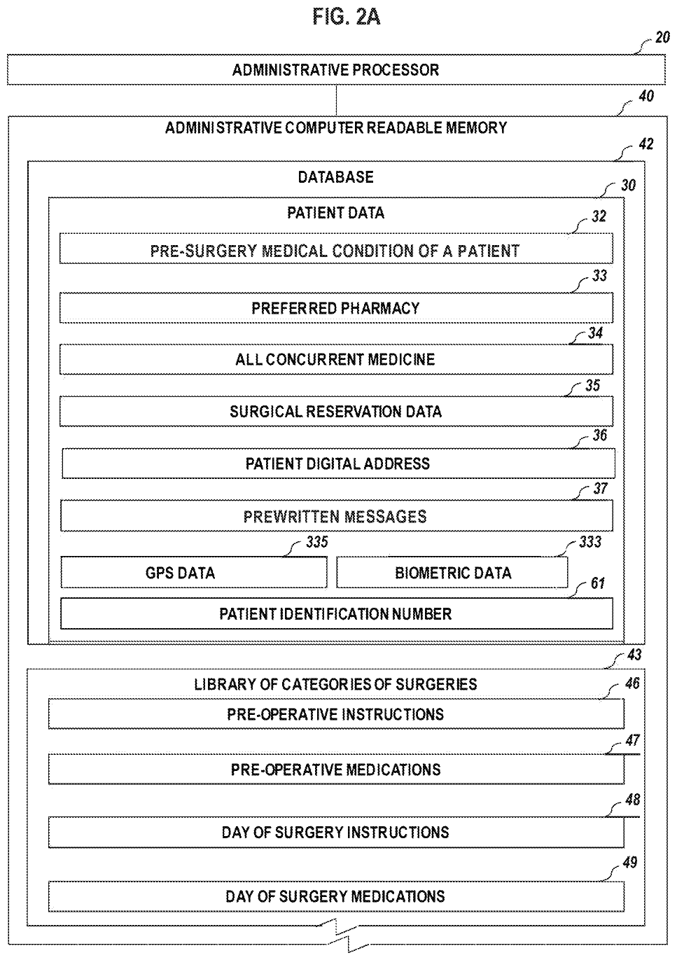

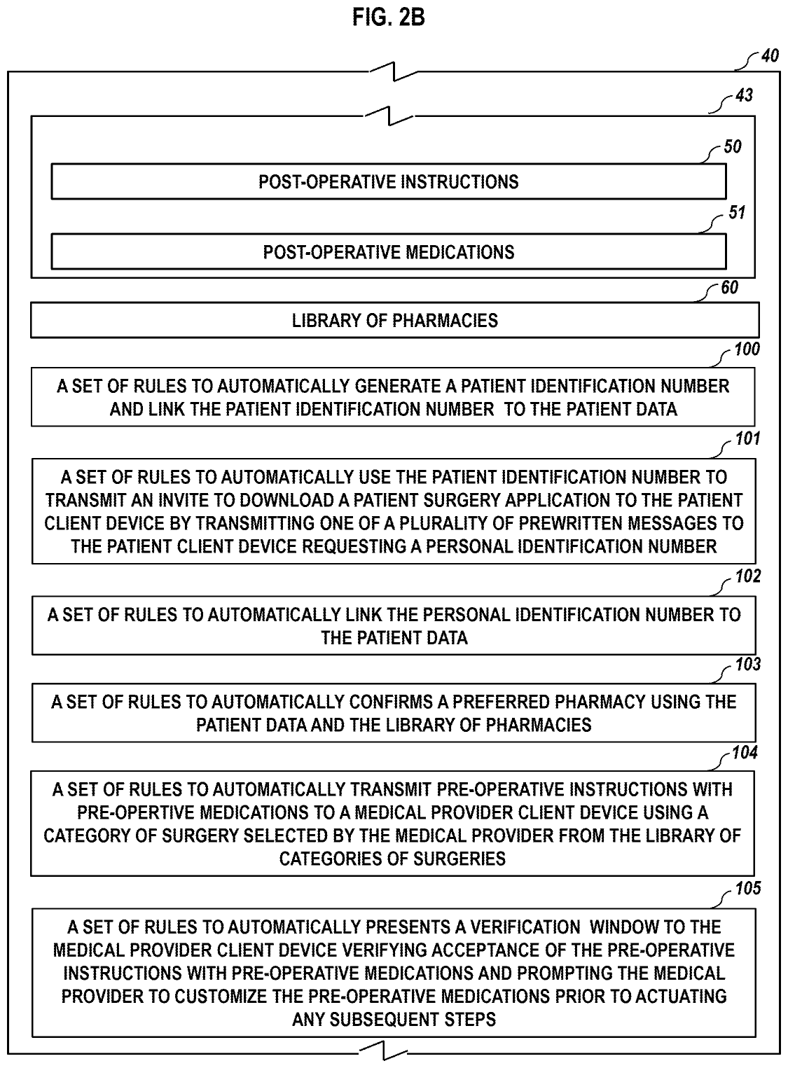

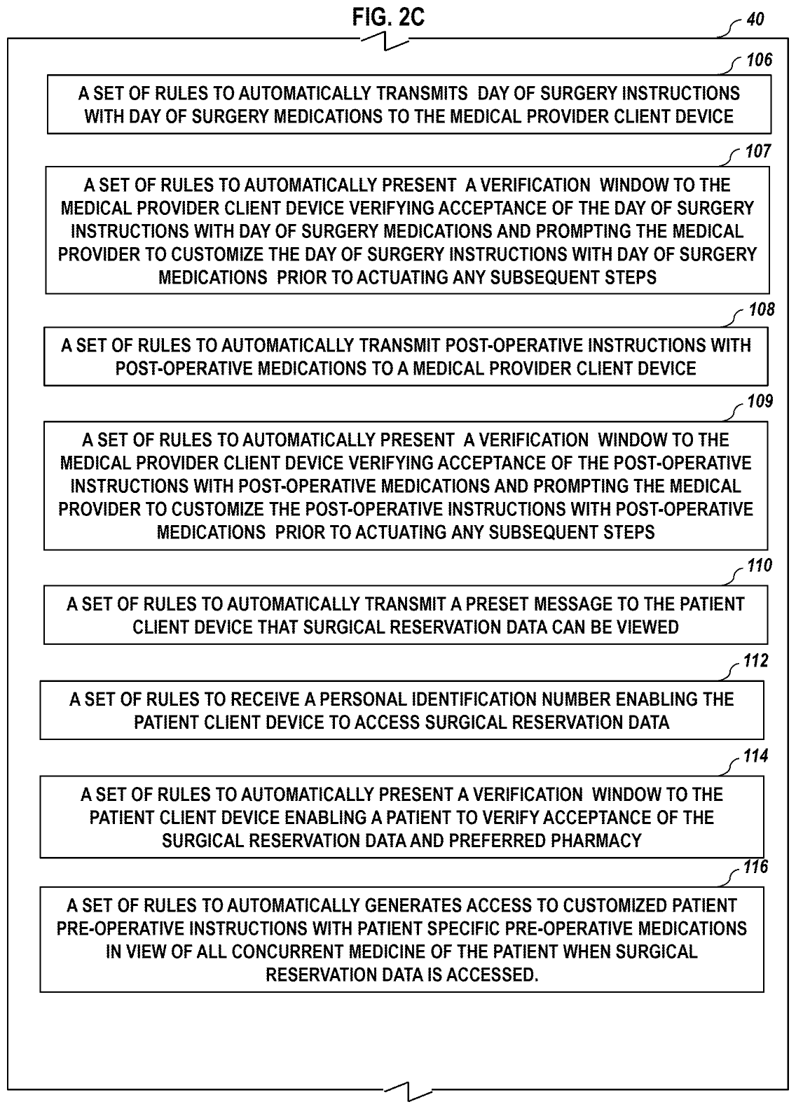

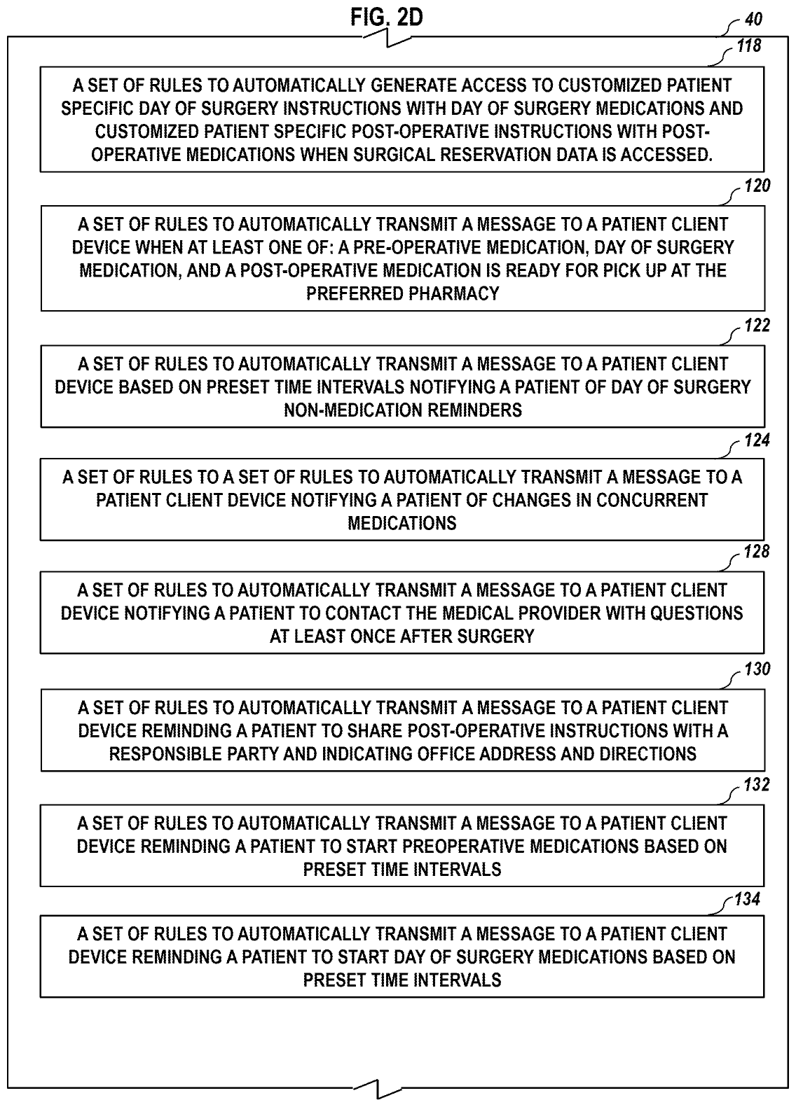

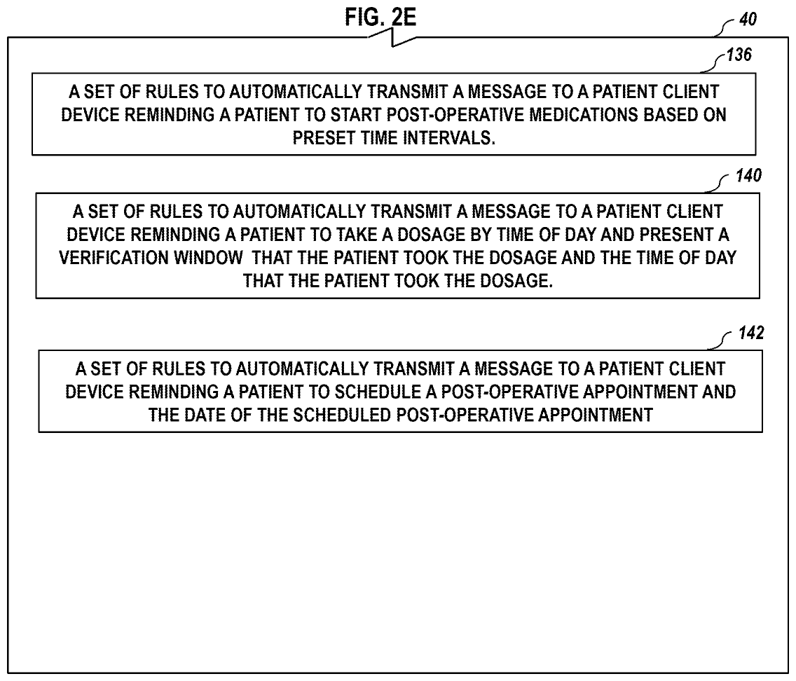

FIGS. 2A-2E depict a diagram of the administrative computer readable memory 40 according to the invention.

The administrative processor 20 is in communication with the administrative computer readable memory 40, such as a PC with 5 terabytes of memory. The administrative processor can be a computer, and Ipad, a plurality of connected computers or other processors identified in the definitions section of the application.

The administrative computer readable memory 40 includes a database 42 for, containing the patient data 30. The database 42 can be a Sybase-iSQL database.

The patient data 30 contains a patient identification number 61 as well as preset messages 37, which are transmitted to a patient client device, such as “your surgical appointment is reserved for May 9, 2018” and “click here for more information.” The preset messages advise a patient that surgical reservation data 35 is available for viewing.

The patient data 30 contains applicable pre-surgery medical conditions of a patient 32, such as congestive heart failure which are transmitted to the medical provider client device to ensure the patient preexisting conditions are viewed by the medical provider using the medical provider device.

The patient data 30 includes a preferred pharmacy 33, such as a CVS™ at a certain geographic location (address).

The patient data 30 contains all concurrent medicine 34 that is being taken by the patient, such as LISINOPRIL™. The method contemplates providing medicines and pre-existing medical conditions of a patient to a medical provider device.

The patient data 30 includes surgical reservation data 35, such as “May 9, 2018 at 7:30 am at 6400 Fannin Street, Suite 1500, Houston, Tex., 77030 for tooth extraction”. The same surgical reservation data is transmitted using the method from the administrative processor to both the patient client device and the medical provider client device for automatic insertion into the calendars of both client devices.

The patient data 30 includes a patient digital address 36, such as an actual address of 7137 Candy Lane, Houston Tex. or an email address such as BettyBoop@gmail.com.

The patient data include GPS data 335 and patient and medical provider biometric data 333.

The administrative computer readable memory 40 includes a library of categories of surgeries 43.

The library of categories of surgeries 43 includes pre-operative instructions 46, such as start “an antibiotic 24 hours before surgery”.

The library of categories of surgeries 43 includes pre-operative medications 47, such as “the antibiotic known as CLARITHROMYCIN™” with instructions to start medication 12 hours before surgery.

The library of categories of surgeries 43 includes day of surgery instructions 48, such as, no eating 6 hours before surgery.

The Library of categories of surgeries 43 includes day of surgery medications 49, such as TYLENOL 3™ with instructions to start medication 4 to 6 hours after surgery.

The Library of categories of surgeries 43 includes post-operative instructions 50, such as start taking a steroid 24 hours after surgery, refrain from nose blowing, refrain from sucking straws, and eat only soft foods for 48 hours.

The Library of categories of surgeries 43 includes post-operative medications 51, such as a MEDROL DOSE PAK™ 5 mg with instructions to start medication 12 hours after surgery.

A Library of pharmacies 60 is in the administrative computer readable memory and includes names of pharmacies, such as “Tschiffely pharmacy”, addresses of pharmacies, such as 1145 19th Street, NW Wash., DC, 20036, hours of operation of the pharmacy, such as 8:30 am-6 pm; dates the pharmacy is closed, such as Saturday and Sunday, phone numbers of the pharmacist and the pharmacy in general such as 202-466-8440, directions to reach the pharmacy, website of the pharmacy, ratings of the pharmacy, and reviews such as from other patients on pharmacy cleanliness, service, and quality of stock.

In the administrative computer readable media 40 are a plurality of rules, namely, rules to: (a) identification rules 100 that cause the administrative processor to automatically generate a patient identification number 61 and link the patient identification number to the patent data 30; (b) invitation rules 101 that cause the administrative processor to automatically use the patient identification number to transmit an invite to a surgery to a patient surgery application 99 that has been downloaded into the patient client device, wherein the invitation is in the form of one of a plurality of prewritten messages 97 requesting a personal identification number 65; (c) matching rules 102 that cause the administrative processor to automatically link the personal identification number 65 to the patient data 30; (d) preferred pharmacy rules 103 that cause the administrative processor to automatically confirm a preferred pharmacy 33 of the patient's choice using the patient data 30 and the library of pharmacies 60 with the patient client device; (e) preoperative instructions rules 104 that cause the administrative processor to automatically transmit pre-operative instructions 46 with pre-operative medications 47 from the Library to the medical provider client device 23 using a category of surgery selected by the medical provider from the library of categories of surgeries; (f) first verification rules 105 that cause the administrative processor to automatically present a first verification window 72a to the medical provider client device verifying acceptance of the selected pre-operative instructions with pre-operative medications and prompting the medical provider to customize the pre-operative instructions with pre-operative medications prior to actuating any subsequent steps; (g) day of surgery rules 106 that cause the administrative processor to automatically transmit day of surgery instructions 48 with day of surgery medications 49 to the medical provider client device; (h) second verification rules 107 that cause the administrative processor to automatically present a second verification window 72b to the medical provider client device requesting acceptance of the day of surgery instructions with day of surgery medications and requesting the medical provider to customize the day of surgery instructions with day of surgery medications prior to actuating any subsequent steps; (i) post operative rules 108 that cause the administrative processor to automatically transmit post-operative instructions 50 with post-operative medications 51 to a medical provider client device; (j) third verification rules 109 that cause the administrative processor to automatically present a third verification window 72c to the medical provider client device requesting acceptance of the post-operative instructions with post-operative medications and requesting the medical provider to customize the post-operative instructions with post-operative medications prior to actuating any subsequent steps; (k) preset message transmission rules 110 that cause the administrative processor to automatically transmit a preset message 37 stored in the Library of Category of Surgeries to the patient client device that surgical reservation data 35 can be viewed; (1) personal identification rules 112 that cause the administrative processor to receive and store a personal identification number 65 enabling the patient client device to access surgical reservation data 35; and (m) fourth verification rules 114 that cause the administrative processor to automatically present a third verification window 72d to the patient client device enabling a patient to verify acceptance of the surgical reservation data 35 and preferred pharmacy.

In embodiments, the method uses a set of pre-operative customization rules 116 to cause the administrative processor to automatically generate access to patient pre-operative instructions 62 with patient specific pre-operative medications 63 and customize pre-operative instructions in view of all concurrent medicine 34 of the patient when surgical reservation data is accessed.

In embodiments, the method uses a set of day of surgery rules 118 to cause the administrative processor to automatically suggest the medical provider to customize patient specific day of surgery instructions with day of surgery medications 64; and enable customization of patient specific post-operative instructions with post-operative medications 66 when surgical reservation data is accessed.

In embodiments, the method uses a set of “medication is ready” rules 120 to instruct the administrative processor to automatically transmit a message to at least one patient client device when at least one of: a pre-operative medication, day of surgery medication and a post-operative medication is ready for pick up at the preferred pharmacy.

In embodiments, the method uses a set of non-surgery preoperative reminder rules 122 to cause the administrative processor to automatically transmit a message to a patient client device based on preset time intervals notifying a patient of day of surgery non-medication activity.

In embodiments, the method uses a set of changes in concurrent medication rules 124 to cause the administrative processor to automatically transmit a message to a patient client device notifying a patient of changes in concurrent medications.

In embodiments, the method uses a set of “need to contact medical provider” rules 128 to cause the administrative processor to automatically transmit a message to a patient client device notifying a patient to immediately contact the medical provider with questions after surgery.

In embodiments, the method uses a set of “responsible party” rules 130 to cause the administrative processor to automatically transmit a message to a patient client device reminding a patient to share post-operative instructions with a responsible party.

In embodiments, the method uses a set of “start pre-operative medication” rules 132 to cause the administrative processor to automatically transmit a message to a patient client device reminding a patient to start pre-operative medications based on preset time intervals saved in the memory of the administrative processor.

In embodiments, the method uses a set of “transmit a message on day of surgery” rules 134 to cause the administrative processor to automatically transmit a message to a patient client device reminding a patient to start day of surgery medications based on preset time intervals saved in the memory of the administrative processor.

The method utilizes a set of post-operative medication rules 136 to cause the administrative processor to automatically transmit a message to a patient client device reminding a patient to start post-operative medications based on preset time intervals saved in the memory of the administrative processor.

The method utilizes a set of dosage verification rules 140 to cause the administrative processor to automatically transmit a message to a patient client device reminding a patient to take a dosage of a certain medication by time of day and to present a verification window to the patient client device enabling the patient to verify that the patient took the dosage of the medication and the time of day that the patient took the dosage.

Biometric files can be used in embodiments to connect the medical provider client devices to the files stored in the medical provider client device and to provide a double authentication with the administrative server using stored biometric files in the computer readable memory used with this method

The term “biometric files” can include fingerprint recognition, iris recognition, facial recognition, hand geometry recognition and voice recognition. The administrative processor can use ‘Knomi software by Aware manufacturing of the USA for creating and matching the voice and face biometric files. Thales also provides software for biometric creation and matching files.

GPS features of the patient client device and the medical provider device can be used along with software applications such as “Find my Friend” loaded into the patient client device to enable the method to use similar “Find my Friend” software and specialty software to match when the patient arrives at the surgical location on the day of surgery for “touch free” check in on day of surgery.

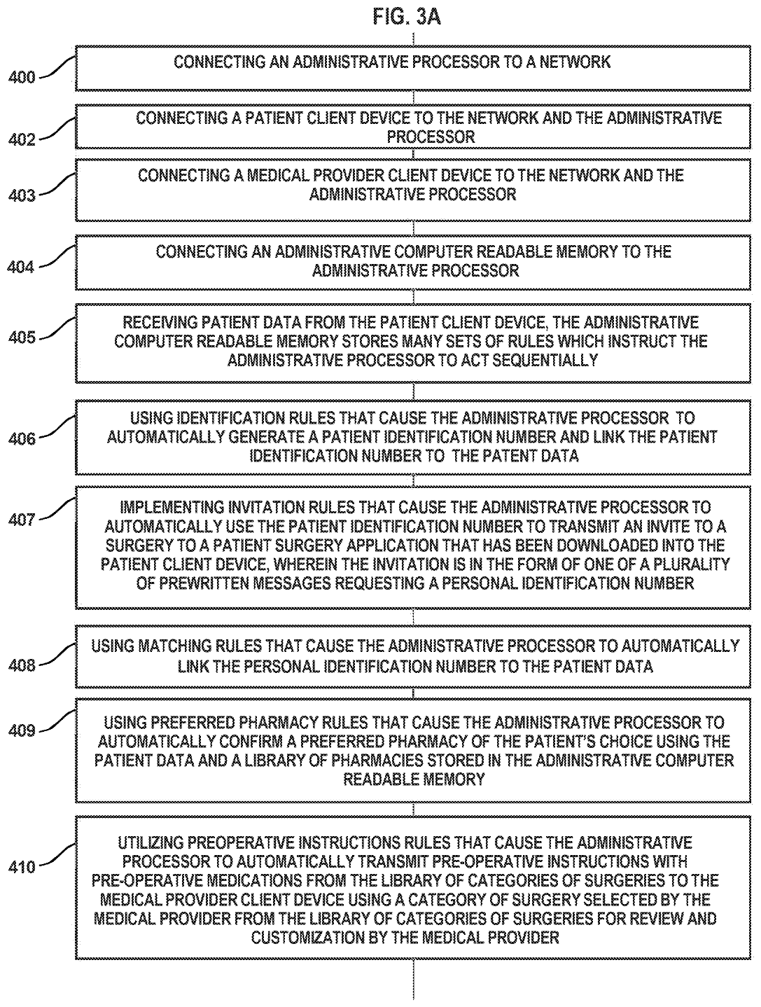

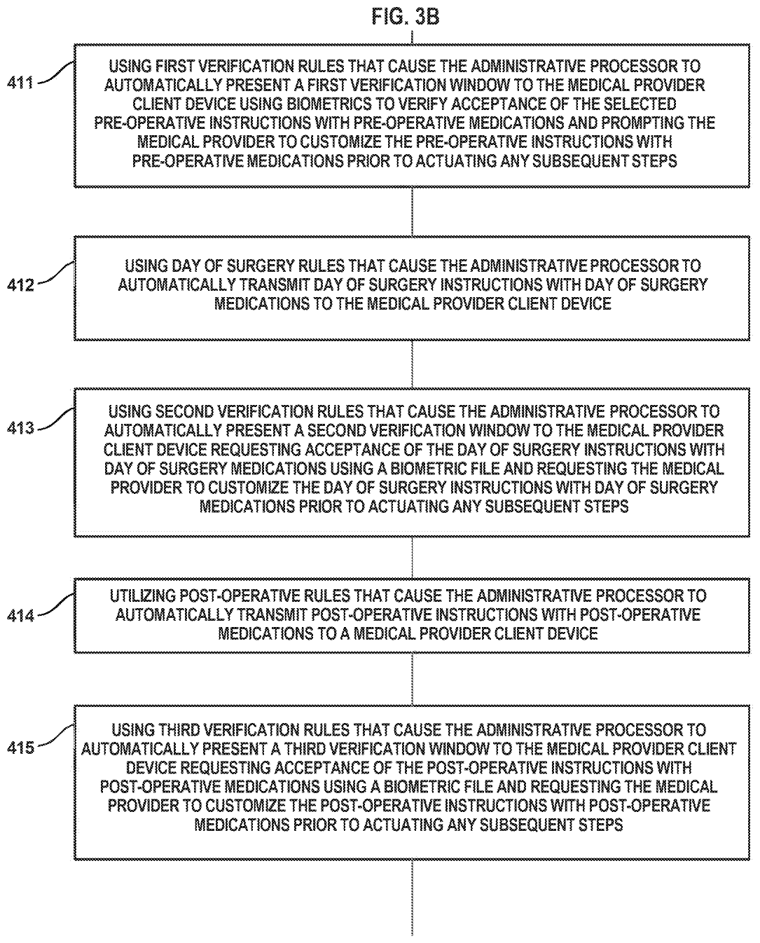

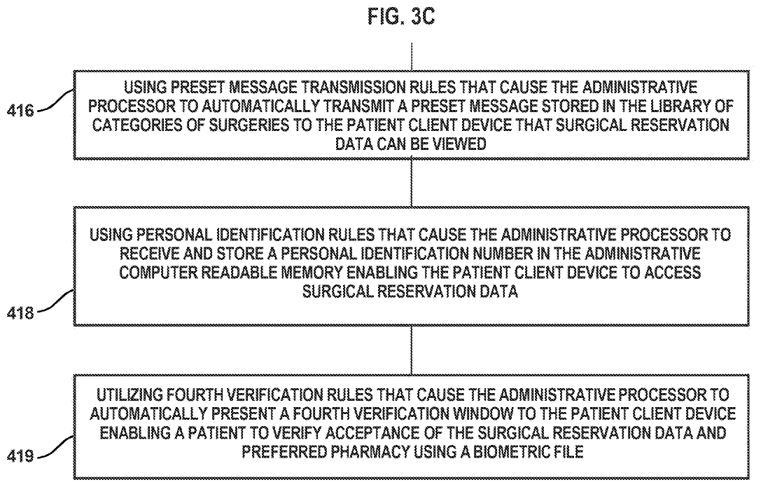

FIGS. 3A-3C presents embodiments of steps of the invention.

Step 400 involves connecting an administrative processor to a network.

Step 402 involves connecting a patient client device to the network and the administrative processor.

Step 403 involves connecting a medical provider client device to the network and the administrative processor.

Step 404 involves connecting an administrative computer readable memory to the administrative processor.

Step 405 involves receiving patient data from the patient client device. The administrative computer readable memory stores many sets of rules which instruct the administrative processor to act sequentially.

Step 406 involves using identification rules that cause the administrative processor to automatically generate a patient identification number and link the patient identification number to the patent data.

Step 407 is the step of implementing invitation rules that cause the administrative processor to automatically use the patient identification number to transmit an invite to a surgery to a patient surgery application that has been downloaded into the patient client device, wherein the invitation is in the form of one of a plurality of prewritten messages requesting a personal identification number.

Step 408 involves using matching rules that cause the administrative processor to automatically link the personal identification number to the patient data.

Step 409 involves using preferred pharmacy rules that cause the administrative processor to automatically confirm a preferred pharmacy of the patient's choice using the patient data and a Library of Pharmacies stored in the administrative computer readable memory.

Step 410 involves utilizing preoperative instructions rules that cause the administrative processor to automatically transmit pre-operative instructions with pre-operative medications from the Library of Categories of Surgeries to the medical provider client device using a Category of Surgery selected by the medical provider from the Library of Categories of Surgeries for review and customization by the medical provider.

Step 411 involves using first verification rules that cause the administrative processor to automatically present a first verification window to the medical provider client device using biometrics to verify acceptance of the selected pre-operative instructions with pre-operative medications and prompting the medical provider to customize the pre-operative instructions with pre-operative medications prior to actuating any subsequent steps

Step 412 involves using day of surgery rules that cause the administrative processor to automatically transmit day of surgery instructions with day of surgery medications to the medical provider client device.

Step 413 involves using second verification rules that cause the administrative processor to automatically present a second verification window to the medical provider client device requesting acceptance of the day of surgery instructions with day of surgery medications using a biometric file and requesting the medical provider to customize the day of surgery instructions with day of surgery medications prior to actuating any subsequent steps.

Step 414 utilizes post-operative rules that cause the administrative processor to automatically transmit post-operative instructions with post-operative medications to a medical provider client device.

Step 415 involves using third verification rules that cause the administrative processor to automatically present a third verification window to the medical provider client device requesting acceptance of the post-operative instructions with post-operative medications using a biometric file and requesting the medical provider to customize the post-operative instructions with post-operative medications prior to actuating any subsequent steps.

Step 416 involves using preset message transmission rules that cause the administrative processor to automatically transmit a preset message stored in the Library of Categories of Surgery to the patient client device that surgical reservation data can be viewed.

Step 418 involves using personal identification rules that cause the administrative processor to receive and store a personal identification number in the administrative computer readable memory enabling the patient client device to access surgical reservation data.

Step 419 involves utilizing fourth verification rules that cause the administrative processor to automatically present a fourth verification window to the patient client device enabling a patient to verify acceptance of the surgical reservation data and preferred pharmacy using a biometric file.

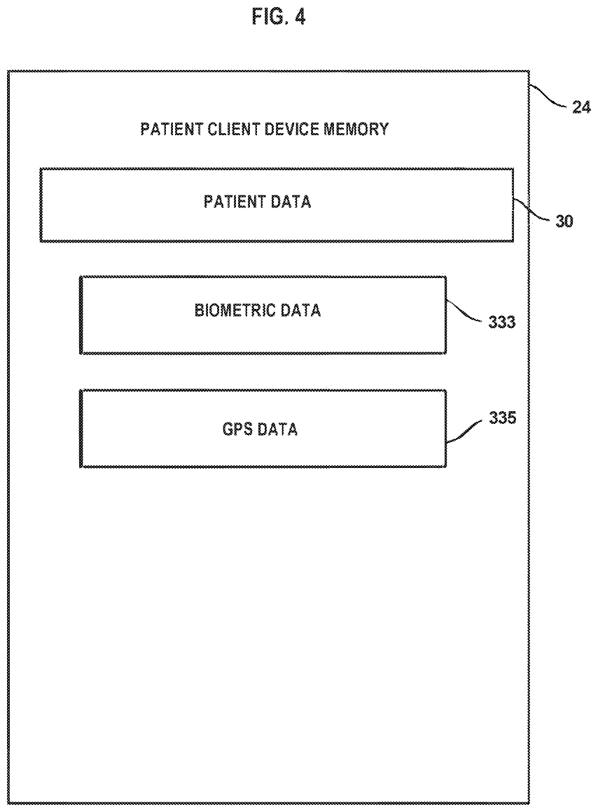

FIG. 4 shows a Patient Client Device memory 24 with patient data 30, Biometric data 333 and GPS data 335 contained therein.

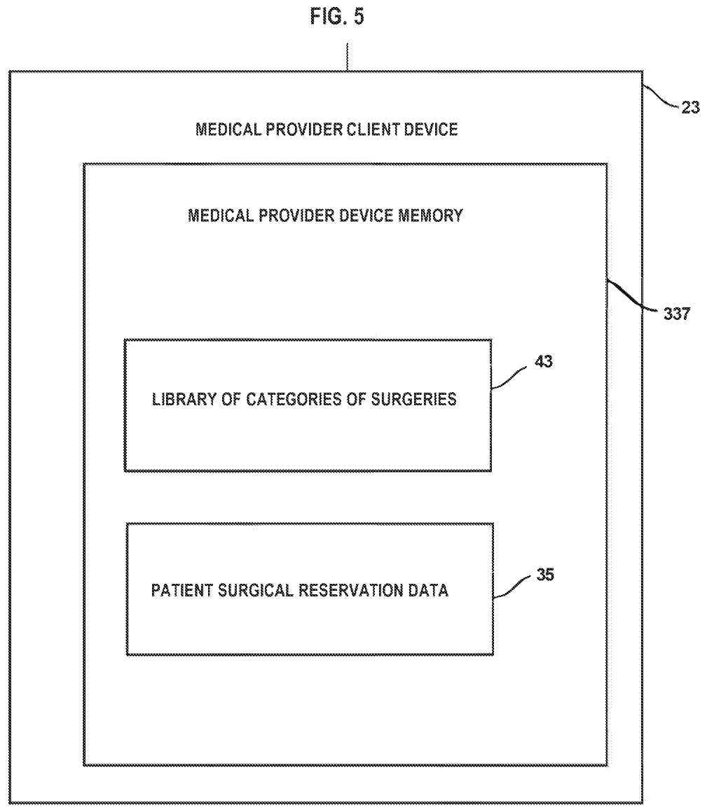

FIG. 5 shows a medical provider client device 23 with a medical provider device memory 337 a Library of Categories of Surgeries 43 and Patient surgical reservation data 35.

### EXAMPLE 1

A method for use oral surgery office a group of patients having oral surgery.

The surgeon's office connects an administrative processor connected to a global communication network like the Internet.

Patients can connect to use the method using a smart phone as their patient client device to receive and transmit information bi-directionally to the surgeon's office and the administrative processor simultaneously.

The patient provides patient data to a medical provider client device at the doctor's office, such as Dr. Brownfield.

For example, for Mary, age 32 needs wisdom teeth removed. Mary would input her applicable pre-surgery medical condition, such as she had a miscarriage 3 months prior to the surgery, her weight, 130 pounds, and other details on medical history. Mary inputs her patient digital address, such as an email. She inputs that her preferred pharmacy is WALGREENS™ at the corner of Montrose and Hawthorne.

Mary inputs that her concurrent medicine is birth control and SYNTHROID™.

After being viewed by the medical provider and deciding on a surgery, the medical provider inputs to Mary's patient data, surgical reservation data including the type of surgery, the day of surgery, the time of surgery, the location of the surgery, the length of time expected for the surgery and other details.

In the administrative computer readable memory is a database containing the patient data and a library of categories of surgeries including the category of surgery for wisdom teeth removal.

The method for Mary uses logic and a set of rules to: automatically generate a patient identification number for Mary, such as MaryC1345, and then link MaryC1345 to her patient data.

The method for Mary uses logic and a set of rules to automatically confirm a preferred pharmacy, the Walgreens at Montrose and Hawthorne using Mary's patient data and the library of pharmacies.

The method for Mary uses logic and a set of rules to automatically transmit pre-operative instructions with pre-operative medications to her Doctor Brownfield's medical provider client device using a category of surgery selected by the medical provider from the library of categories of surgeries.

The method for Mary uses logic and a set of rules to automatically present to Dr. Brownfield a verification window on her office laptop verifying acceptance of the pre-operative instructions with pre-operative medications and prompting the medical provider to customize the pre-operative instructions with pre-operative medications prior to actuating any subsequent steps.

The method for Mary uses logic and a set of rules to automatically transmit prior to and on the day of surgery very specific day of surgery instructions with day of surgery medications to Dr. Brownfield for the Doctor to confirm as correct or to modify given patient data.

The method for Mary uses logic and a set of rules to automatically present a verification window to Dr. Brownfield's office computer to verify acceptance of the day of surgery instructions with day of surgery medications and prompt the medical provider to customize the day of surgery instructions with day of surgery medications prior to actuating any subsequent steps.

The method for Mary uses logic and a set of rules to automatically transmit post-operative instructions with post-operative medications to Dr. Brownfield and automatically present a verification window to the medical provider client device verifying acceptance of the post-operative instructions with post-operative medications and prompting the medical provider to customize the post-operative instructions with post-operative medications prior to actuating any subsequent steps.

The method for Mary uses logic and a set of rules to automatically use the patient identification number such as: MaryC1345 to transmit a message to download a patient surgery application from the administrative computer readable memory to the patient client device by transmitting one of a plurality of prewritten messages to the patient client device requesting a personal identification number.

The patient surgery application is resident in administrative computer readable memory connected to the administrative processor. Portions of the patient surgery application can be downloaded to Mary's cell phone.

Mary transmits a personal identification number BettyBoop1 to the administrative processor enabling Mary to access surgical reservation data on her cell phone.

The method for Mary uses logic and a set of rules to automatically link the personal identification number BettyBoop1 to the patient data of Mary.

The method for Mary uses logic and a set of rules to automatically transmit a preset message to Mary's cell phone that surgical reservation data can be viewed.

It should be noted that the method automatically presents a verification window to Mary's cell phone enabling a patient to verify acceptance of the surgical reservation data and preferred pharmacy.

Automatically, after Mary confirms her surgery reservation and her preferred pharmacy, the administrative processor will use logic to operate on a set of rules to provide very specific wisdom teeth removal pre-operative instructions with wisdom teeth removal pre-operative medications. Mary is told to go to the preferred pharmacy and obtain AMOXICILLIN™ which has been automatically transmitted to the preferred pharmacy by the administrative processing using patient data.

Before the surgery, Mary will receive on her patient client device day of surgery instructions with day of surgery medications.

The message would arrive at least 24 hours before the surgery. This message could include the address (such as 90 Main street Houston, Tex.), the time, such as 2 pm, and the geographic collection point, such as East Entrance, to collect the patient after surgery, and the option to share this information with Mary's responsible party.

Additionally after the surgery or day of surgery Mary will get post-operative instructions with post-operative medications. The post-operative instructions will include interactive acknowledgement of the instructions on a step by step basis to ensure safety of the patient and quick recovery with minimal complications, as well as the option to share post-operative instructions and post-operative medications with Mary's responsible party.

The method uses logic to utilize a set of rules to automatically transmit a message to Mary's patient client device reminding Mary to start post-operative medications based on preset time intervals, such as “Take first dose now” and the time is 12 hours following surgery.

Not only does the method remind Mary to take a dosage by time of day, but the method presents a verification window to Mary's cell phone enabling Mary to verify that she took her specific dosage of post-operative medication on a specific day and at a specific time.

The method reminds Mary to repeat the last two steps of reminding and verifying the taking of the post-operative dosage amounts until the post-operative medication is no longer needed.

### EXAMPLE 2

The following is an example of the touchless and automatic method to initiate, track and control with verifications, biometrics and GPS, pre-operative surgical procedures and activities, day of surgery procedures and activities, and post-operative surgical procedures and activities.

In this Example, the surgeon, Dr. Brown's, has a medical provider client device that connects to an administrative processor connected to a network like the Internet.

The patients of Dr. Brown can then connect using their patient client device such as their smart phone to receive and transmit their patient information bi-directionally to and from their smart phone. The connection not only enable the transmission of patient data but enables the transmission of instructions to the patient client device such as pre-operative instructions to stop Aspirin 5 days prior to surgery from the administrative server based on an indication of the type of surgery the patient is to undertake from the medical providers client device and applying rules on pre-operative procedures.

In this Example, the patient data can be applicable pre-surgery medical condition of a patient such Type I Diabetic; a preferred pharmacy such as Walgreens™; a list of all concurrent medicine such as Novolog™ Insulin, Levemir™ Insulin, Fosinipril™ and Jardiance™; surgical reservation data such as an address of the “Mid Town Surgical Center” located at 2105 Jackson St. #200, Houston, Tex. 77003 on a specific date, like Dec. 11, 2020 at a specific time, 9 am; and the patient data must include a patient digital address such as her email.

A medical provider client device at Dr. Brown's office which can be Dr. Browns smart phone is connected to the network such as through a cellular connection to transmit information bi-directionally to and from the administrative processor to input surgical reservation data and receive confirmation of surgical reservation data.

In this Example, the administrative computer readable memory contains a Library of Categories of Surgeries such as information on a “cholecysetectomy.”

Each category of surgery in the Library of Categories of Surgeries has pre-operative instructions such as . . . “no dinner the night prior to the surgery” and with pre-operative medications such as Amoxicillin™.

Each category in the Library of Categories of Surgeries has day of surgery instructions, such as no food or drink 4 hours prior to surgery. Each category in the Library of Categories of Surgeries has a list of day of surgery medications such as Lorazepam™ for procedure anxiety.

Each category in the Library of Categories of Surgeries has post-operative instructions such as “rest” and has post-operative medications, such as Vicodin™ for pain management.

The computer readable administrative memory also has a Library of Pharmacies such as CVS™ located at the corner of Montrose and Richmond.

In this Example, the computer readable memory stores many sets of rules and cause the administrative processor to utilize many sets of rules to specifically do the following: automatically generate a patient identification number such as Bob500 and link the patient identification number to the patent's data; automatically use the patient identification number such as Bob500 to transmit an invite to download a patient surgery application to the patient client device such as smart phone by transmitting one of a plurality of prewritten messages such as “please download the mobile device application and select a personal identification number for log in security” to the patient client device requesting a personal identification number.

In this Example, the computer readable memory uses stored rules to automatically confirms a preferred pharmacy such as WALGREENS™ at Westheimer and Avenue Q using the patient data and the Library of Pharmacies.

In this Example, the computer readable memory uses stored rules to automatically transmits pre-operative instructions such as stopping Omega-3 intake 7 days prior to surgery with pre-operative medications which can include Valium™ to a medical provider client device such as “a Dr. Brown's office CPU” using a category of surgery selected by the medical provider from the Library of Categories of Surgeries.

In this Example, the computer readable memory uses stored rules to automatically presents a verification window to Dr. Brown's medical provider client device verifying acceptance of the pre-operative instructions with pre-operative medications and prompting the medical provider to customize the pre-operative instructions with pre-operative medications prior to actuating any subsequent steps.

In this Example, the computer readable memory uses stored rules to automatically transmit day of surgery instructions such as “. . . do not to take Tums the day of surgery with day of surgery medications” . . . such as Clindamycin™ to the Dr Brown's device, see Set of Rules 106.

In this Example, the computer readable memory uses stored rules automatically to present a verification window to Dr. Brown's device verifying acceptance of the day of surgery instructions with day of surgery medications and prompting the medical provider to customize the day of surgery instructions with day of surgery medications prior to actuating any subsequent steps.

In this Example, the computer readable memory uses stored rules to automatically transmit post-operative instructions such as liquid diet for the first day with post-operative medications such as Advil™ 800 mg e 6 hours as needed for post-operative discomfort to Dr. Brown's device;

In this Example, the computer readable memory uses stored rules enabling Dr. Brown to automatically received a verification window to his office CPU to verify acceptance of the post-operative instructions with post-operative medications and prompting Dr. Brown to customize the post-operative instructions with post-operative medications prior to actuating any subsequent steps. Dr. Brown modifies post-operative instruction to a liquid diet for the first 2 days and post-operative medication to Tylenol™ 1000 mg every 6 hours for the first 5 days.

In this Example, the computer readable memory uses stored rules to automatically transmit a preset message to the patient, Bob's device that surgical reservation data such as confirmation of surgical reservation location and time available.

In this Example, the computer readable memory uses stored rules to Bob input's his personal identification number Bob500 enabling his patient client device to access surgical reservation data such as the name address and date of the surgery , such as Mid Town Surgical Center at 2105 Jackson St. #200, Houston, Tex. 77003 on Dec. 11, 2020 at 9 am.

In this Example, the computer readable memory uses stored rules enabling Bob to see a verification window on his smart phone enabling him to verify acceptance of the surgical reservation data and preferred pharmacy such as CVS™ at the corner of Montrose and Richmond, see Rule 114.

In this Example, the method has contemplated using a set of rules stored in the administrative computer readable memory to automatically generates access for the office of Dr Brown to customize patient pre-operative instructions such as to stop the patient's baby aspirin 5 days prior to surgery with patient specific pre-operative medications such as to start Amoxicillin™ 500 mg TID 3 days prior to surgery in view of all concurrent medicine such as Novolog™ Insulin, Levemir™ Insulin, and Jardiance™ currently being taken by the patient.

In this Example, the method has contemplated using a set of rules stored in the administrative computer readable memory to use the set of rules 118 to automatically generate access to customize patient specific day of surgery instructions such as not taking or lower insulin medication the day of surgery with day of surgery medications to include Bob's morning dosage of Amoxicllin™ prior to surgery; and customize patient specific post-operative instructions such as liquid diet for the first 2 days with post-operative medications such as Tylenol™ 1000 mg every 6 hours when surgical reservation data is accessed.

In this Example, the method has contemplated using a set of rules stored in the administrative computer readable memory to set of rules 120 to automatically transmit a message to Bob's smart phone when at least one of: a pre-operative medication, day of surgery medication, and a post-operative medication is ready for pick up at the preferred pharmacy.

In this Example the method has contemplated using a set of rules 122 stored in the administrative computer readable memory to automatically transmit a message to a Bob's smart phone based on preset time intervals notifying Bob of day of surgery non-medication reminders such as no eating or drinking the day of surgery.

In this Example the method has contemplated using a set of rules 124 stored in the administrative computer readable memory to automatically transmit a message to Bob's device notifying a patient of changes in concurrent medications such as stopping Insulin prior to surgery.

In this Example, the method has contemplated using a set of rules 128 stored in the administrative computer readable memory to automatically transmit a message to Bob's device notifying him to contact the medical provider with questions at least once after surgery, such as a note from Dr. Brown's office stating that if you have any questions or concerns in regard to your post-operative instruction please contact us now.

In this Example, the method has contemplated using a set of rules 130 stored in the administrative computer readable memory to automatically transmit a message to a Bob' device reminding him to share post-operative instructions with a responsible party, such as click here and enter email of the responsible party you would like to share your post-operative instructions with.

In this Example, the method has contemplated using a set of rules stored in the administrative computer readable memory to The system of claim 1, wherein the computer readable memory stores logic to utilize a set of rules 132 to automatically transmit a message to Bob's device reminding him to start preoperative medications based on preset time intervals, such as “Your surgery is in 2 days. Today you should start your Amoxicillin.”

In this Example, the method has contemplated using a set of rules 134 stored in the administrative computer readable memory to to automatically transmit a message to Bob's device reminding him to start day of surgery medications based on preset time intervals, such as Bob you are due to take your next does of Amoxicillin™ now, 8 am.

In this Example, the method has contemplated using a set of rules 136 stored in the administrative computer readable memory to automatically transmit a message to Bob's device reminding him to start post-operative medications based on preset time intervals, such as “Our records indicate you should be taking your next dosage of Tylenol™ 100 mg now, 4 pm”.

In this Example, the method has contemplated using a set of rules 140 stored in the administrative computer readable memory to automatically transmit a message to a Bob's device reminding him to take a dosage by time of day and present a verification window that the patient took the dosage and the time of day that the patient took the dosage such as “Our records indicate that you are due for 2^{nd} dosage today of Amoxicillin™ 500 mg now, 12 pm. Please click here that you have taken your 2^{nd} dosage of Amoxicillin™ 500 mg, and enter the time of day this dosage was taken”.

While these embodiments have been described with emphasis on the embodiments, it should be understood that within the scope of the appended claims, the embodiments might be practiced other than as specifically described herein.

What is claimed is:

1. A touchless and automatic method to set up a plurality of patients for surgery simultaneously by touchlessly combining patient data 30 and instructions and verification of instructions using biometric data for preoperative procedures including pre-operative medication 47, GPS data for touchless day of surgery check in 38, instructions and verification of instructions using biometric data 39 concerning day of surgery medication, and instructions and verification of instructions using biometric data concerning post-operative procedures including post-operative medication, the method comprising:
a. connecting an administrative processor 20 to a network 21;
b. connecting a patient client device 22 to the network and the administrative processor 20, the patient client device having a patient client device memory 24 configured to:
i. receive and store patient data 30 including patient name, patient identification number, a preferred pharmacy 33 of a patient, pre-surgery medical condition of a patient 32; patient concurrent medicine 34; patient surgical reservation data 35; and a patient digital address 36 of a patient;
ii. receive and store instructions to receive and transmit information bi-directionally patient data 30 including biometric data 333 of the patient and GPS data 335 from the patient client device to the administrative processor;
c. connecting a medical provider client device 23 to the network 21 and the administrative processor 20, the medical provider client device having a meical provider client device memory 337 configured to:
receive store and update a Library of Categories of Surgery 43;
(ii) receive and store patient surgical reservation data 35, and
(iii) receive and store instructions to receive patient data and customize automatically built instructions by the administrative processor for transmission to the patient client device;
d. connecting an administrative computer readable memory 40 to the administrative processor 20, the administrative computer readable memory comprising:
(i) patient data 30 consisting of patient identification number 31, a preferred pharmacy 33 of a patient, pre-surgery medical condition of a patient 32; patient concurrent medicine 34; surgical reservation data 35; and a patient digital address 36 of a patient;
(ii) a Library of Categories of Surgeries 43, each Category of Surgeries comprising:
(a) pre-operative instructions 46 with pre-operative medications 47;
(b) day of surgery instructions 48 with day of surgery medications 49;
(c) post-operative instructions 50 with post-operative medications 51; and
(d) a Library of Pharmacies 60;
(e) identification rules 100 that cause the administrative processor to automatically generate a patient identification number 61 and link the patient identification number to the patent data 30;
(f) invitation rules 101 that cause the administrative processor to automatically use the patient identification number to transmit an invite to a surgery to a patient surgery application 99 that has been downloaded into the patient client device, wherein the invitation is in the form of one of a plurality of prewritten messages 97 requesting a personal identification number 65;
(g) matching rules 102 that cause the administrative processor to automatically link the personal identification number 65 to the patient data 30;
(h) preferred pharmacy rules 103 that cause the administrative processor to automatically confirm a preferred pharmacy 33 of the patient's choice using the patient data 30 and the library of pharmacies 60 with the patient client device;
(i) preoperative instructions rules 104 that cause the administrative processor to automatically transmit pre-operative instructions 46 with pre-operative medications 47 from the Library of Categories of Surgeries to the medical provider client device 23 using a Category of Surgery selected by the medical provider from the Library of Categories of Surgeries;
(j) first verification rules 105 that cause the administrative processor to automatically present a first verification window 72a to the medical provider client device using biometrics to verify acceptance of the selected pre-operative instructions with pre-operative medications and prompting the medical provider to customize the pre-operative instructions with pre-operative medications prior to actuating any subsequent steps;
(k) day of surgery rules 106 that cause the administrative processor to automatically transmit day of surgery instructions 48 with day of surgery medications 49 to the medical provider client device; second verification rules 107 that cause the administrative processor to automatically present a second verification window 72b to the medical provider client device requesting acceptance of the day of surgery instructions with day of surgery medications using a biometric file and requesting the medical provider to customize the day of surgery instructions with day of surgery medications prior to actuating any subsequent steps;
(l) post-operative rules 108 that cause the administrative processor to automatically transmit post-operative instructions 50 with post-operative medications 51 to a medical provider client device; third verification rules 109 that cause the administrative processor to automatically present a third verification window 72c to the medical provider client device requesting acceptance of the post-operative instructions with post-operative medications using a biometric file and requesting the medical provider to customize the post-operative instructions with post-operative medications prior to actuating any subsequent steps;
(m) preset message transmission rules 110 that cause the administrative processor to automatically transmit a preset message 37 stored in the Library of Categories of Surgery to the patient client device that surgical reservation data 35 can be viewed;
(n) personal identification rules 112 that cause the administrative processor to receive and store a personal identification number 65 in the administrative computer readable memory enabling the patient client device to access surgical reservation data 35; and fourth verification rules 114 that cause the administrative processor to automatically present a fourth verification window 72d to the patient client device enabling a patient to verify acceptance of the surgical reservation data 35 and preferred pharmacy using a biometric file; and using the method to (i) touchlessly (i) register a patient for surgery, (ii) receive patient data, (ii) build custom messaging to a patient with verification of messaging using biometric data and patient GPS data for (a) preoperative procedures including pre-operative medications, (b) registering a patient for day of surgery, (c) day of surgery procedures, (d) post operative procedures and (e) messaging pharmacies and patients and the medical providers verifying pick up of pre-surgery, day of surgery and post-surgery medications and (f) messaging the patent and medical provider with verification from the patient that a patient is taking the designated medications

2. The method of claim 1 comprising a set of pre-operative customization rules 116 in the administrative processor computer readable memory 40 to cause the administrative processor to automatically generate access to patient pre-operative instructions 62 with patient specific pre-operative medications 63 and customize pre-operative instructions in view of all concurrent medicine 34 of the patient when surgical reservation data is accessed

3. The method of claim 2, comprising a set of day of surgery rules 118 in the computer readable memory to cause the administrative processor to automatically suggest the medical provider customize patient specific day of surgery instructions with day of surgery medications 64; and enable customization of patient specific post-operative instructions with post-operative medications 66 when surgical reservation data is accessed.

4. The method of claim 1, comprising a set of “medication is ready” rules 120 in the computer readable memory to instruct the administrative processor to automatically transmit a message to a patient client device when at least one of: a pre-operative medication, day of surgery medication, and a post-operative medication is ready for pick up at the preferred pharmacy.

5. The method of claim 1, comprising a set of non-surgery preoperative reminder rules 122 in the computer readable memory to instruct the administrative processor to automatically transmit a message to a patient client device based on preset time intervals notifying a patient of day of surgery non-medication activity.

6. The method of claim 1, comprising a set of changes in concurrent medication rules 124 in the computer readable memory to instruct the administrative processor to automatically transmit a message to a patient client device notifying a patient of changes in concurrent medications.

7. The method of claim 1, comprising a set of “need to contact medical provider” rules 128 in the computer readable memory to cause the administrative processor to automatically transmit a message to a patient client device notifying a patient to immediately contact the medical provider with questions after surgery.

8. The method of claim 1, comprising a set of “responsible party” rules 130 in the computer readable memory to cause the administrative processor to automatically transmit a message to a patient client device reminding a patient to share post-operative instructions with a responsible party.

9. The method of claim 1, comprising a set of “start pre-operative medication” rules 132 in the computer readable memory to cause the administrative processor to automatically transmit a message to a patient client device reminding a patient to start pre-operative medications based on preset time intervals.

10. The method of claim 1, comprising a set of “transmit a message on day of surgery” rules 134 in the computer readable memory to cause the administrative processor to automatically transmit a message to a patient client device reminding a patient to start day of surgery medications based on preset time intervals.

11. The method of claim 1, comprising a set of post-operative medication rules 136 in the computer readable memory to cause the administrative processor to automatically transmit a message to a patient client device reminding a patient to start post-operative medications based on preset time intervals.

12. The method of claim 1, comprising a set of dosage verification rules 140 in the computer readable memory to cause the administrative processor to automatically transmit a message to a patient client device reminding a patient to take a dosage of a certain medication by time of day and to present a verification window to the patient client device enabling the patient to verify that the patient took the dosage of the medication and the time of day that the patient took the dosage.
